(19) United States
(12) Patent Application Publication
(10) Pub. No.: US 2008/0032373 A1
(43) Pub. Date: Feb. 7, 2008

(54) Process Of Producing A Fermentation Product

(75) Inventors: Swapnil Bhargava, Raleigh, NC (US); Henrik Frisner, Vaerloese (DK); Henrik Bisgard-Frantzen, Bagsvaerd (DK); Jeppe Wegener Tams, Gentofte (DK)

Correspondence Address: NOVOZYMES NORTH AMERICA, INC., 500 FIFTH AVENUE, SUITE 1600, NEW YORK, NY, 10110, US

(73) Assignees: Novozymes North America, Inc., Franklinton, NC (US); Novozymes A/S., Bagsvaerd (DK)

(21) Appl. No.: 11/579,748

(22) PCT Filed: May 11, 2005
(86) PCT No.: PCT/US05/16390

§ 371 Date: Nov. 8, 2006

Related U.S. Application Data
(60) Provisional application No. 60/570,727, filed on May 13, 2004.
(60) Provisional application No. 60/632,201, filed on Dec. 1, 2004.
(60) Provisional application No. 60/633,293, filed on Dec. 3, 2004.

(51) Int. Cl. C12P 7/14

(52) U.S. Cl. 435/162

(57) ABSTRACT

The invention relates to a process of producing a fermentation product, such as ethanol, from starch-containing material, including i) subjecting starch-containing material to an alpha-amylase, ii) subjecting the material obtained in step i) to an alpha-glucosidase and/or a maltose-generating enzyme, and iii) fermenting the material in the presence of a fermenting organism, such as yeast. Alternatively the invention relates to a process of producing a fermentation product from starch-containing material, preferably granular starch, which process comprises: a) subjecting starch-containing material to an alpha-glucosidase and optionally a glucose-generating and/or maltose-generating enzyme, and b) fermenting the material in the presence of a fermenting organism.

### REFERENCE TO A SEQUENCE LISTING

This application contains a Sequence Listing in computer readable form. The computer readable form is incorporated herein by reference.

### FIELD OF THE INVENTION

The present invention relates to a process for producing a fermentation product, such as ethanol, from starch-containing material.

### BACKGROUND OF THE INVENTION

A vast number of commercial products that are difficult to produce synthetically may be produced by fermentation. Such products including alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones. Fermentation is also commonly used in the consumable alcohol (e.g., beer and wine), dairy (e.g., in the production of yogurt and cheese), leather, and tobacco industries.

Ethanol has widespread application as an industrial chemical, gasoline additive or straight liquid fuel. As a fuel or fuel additive, ethanol dramatically reduces air emissions while improving engine performance. As a renewable fuel, ethanol reduces national dependence on finite and largely foreign fossil fuel sources while decreasing the net accumulation of carbon dioxide in the atmosphere. Fermentation processes are used for the production of ethanol. There are a large number of disclosures concerning production of alcohol by fermentation, among which are, e.g., U.S. Pat. No. 5,231,017, CA 1,143,677, and EP 138428.

There is a need for further improvement of fermentation product, such as ethanol manufacturing processes.

### SUMMARY OF THE INVENTION

The invention relates to processes of producing fermentation products, such as ethanol, from starch-containing material, preferably based on whole grain, said process comprises:

i) subjecting starch-containing material to an alpha-amylase,

ii) subjecting the material obtained in step i) to an alpha-glucosidase and optionally a glucose-generating and/or maltose-generating enzyme, and

iii) fermenting the material in the presence of a fermenting organism.

In a preferred embodiment the alpha-glucosidase is derived from a plant, preferably rice, especially rice (Oryzae sativa).

The present invention also relates to a process of producing a fermentation produced from starch-containing material, which process comprises:

i) subjecting starch-containing material to an alpha-amylase,

ii) subjecting the material obtained in step i) to an alpha-glucosidase and a maltose-generating enzyme, and

iii) fermenting the material in the presence of a fermenting organism.

The fermentation product, such as especially ethanol may optionally be recovered after fermentation, preferably by distillation. Any enzyme having the above mentioned enzyme activities may be used according to the invention. Suitable enzymes are listed in the “Enzyme Activities”-section below. However, in a preferred embodiment the alpha-amylase, preferably bacterial alpha-amylase, used in step i) is derived from the genus Bacillus, especially a strain of Bacillus stearothermophilus or a variant thereof. In a preferred embodiment the maltose-generating enzyme used in step ii) is a maltogenic amylase, especially derived from the genus Bacillus, especially a strain of Bacillus stearothermophilus or a variant thereof. In a preferred embodiment the alpha-glucosidase used in step ii) is of plant, such as especially rice origin, or microbial origin. In the case of the alpha-glucosidase is of bacterial origin, it may preferably be derived from a strain of the genus Bacillus, especially a strain of Bacillus stearothermophilus or a variant thereof. In a preferred embodiment the fermenting organism used in the fermentation step iii) is yeast, preferably of Saccharomyces origin, preferably a strain of Saccharomyces cerevisiae.

The invention also relates to a process of producing a fermentation product, such as ethanol, from starch-containing material, which process comprises:

a) subjecting starch-containing material to an alpha-glucosidase and optionally a glucose-generating and/or maltose-generating enzyme, and

b) fermenting in the presence of a fermenting organism.

In a preferred embodiment the fermentation product is ethanol. In a preferred embodiment the alpha-glucosidase is of rice origin. In a preferred embodiment the starch-containing material is granular starch.

### BRIEF DESCRIPTION OF THE DRAWINGS

FIG. 1 illustrates a process flow diagram for preparing ethanol in accordance with on one embodiment of the invention.

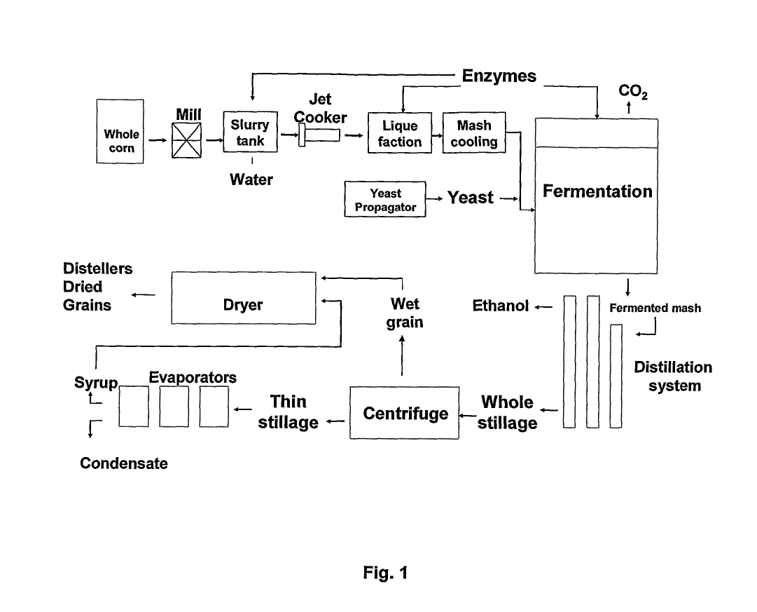

FIG. 2 shows that sugar, glycerol and ethanol profiles for the complete course of SSF for the Reference run.

FIG. 3 shows the sugar, glycerol and ethanol profiles for the complete course of SSF for the Test run.

FIG. 4 shows glucose, DP2, and ethanol profiles for the complete course of SSF for the Test and Reference run plotted in the same graph for easier comparison.

### DETAILED DESCRIPTION OF THE INVENTION

The present invention provides a process for producing a fermentation product especially ethanol, from starch-containing material, which process includes a liquefaction step and separately or simultaneously performed saccharification and fermentation step(s).

The inventors have found that carrying out saccharification and fermentation (especially SSF) in the presence of an effective amount of an alpha-glucosidase and optionally glucose and/or maltose-generating enzyme(s) is advantageous. Without being limited to any theory it is believed that a process of the present invention is more efficient because maltose generated—which is not preferred by yeast in the presence of glucose—is converted to glucose, which is then consumed by the yeast and converted into ethanol. This may lead to a higher fermentation rate and/or a more efficient use of the starch material. Further, the amount of residual sugars after fermentation is reduced. It is further believed that a process of the invention potentially gives the benefit that no or at least less glycero (which cannot be utilized by the yeast) is produced.

Raw Materials

The starch-containing starting material may according to the invention be derived from any plant material. Preferred starting materials are selected from the group consisting of: tubers, roots, whole grain; and any combinations of the forgoing. In one embodiment, the starch-containing material is obtained from cereals. The starch-containing material may, e.g. be selected from the groups consisting of corn, cob, wheat, barley, cassaya, sorghum, rye, milo and potato; or any combination of the forgoing. Wheat and corn are the preferred raw materials.

In a process of the invention, the starch-containing starting material is preferably whole grain or at least mainly whole grain. A wide variety of starch-containing whole grain crops may be used as raw material including: corn (maize), milo, potato, cassaya, sorghum, wheat, and barley.

Thus, in one embodiment, the starch-containing material is whole grain selected from the group consisting of corn (maize), milo, potato, cassaya, sorghum, wheat, and barley; or any combinations thereof. In a preferred embodiment, the starch containing material is whole grain selected from the group consisting of corn, wheat and barley or any combinations thereof.

In one embodiment the starch-containing material is granular starch. The term “granular starch” is understood as raw uncooked starch, i.e., starch that has not been subjected to a gelatinization. Starch is formed in plants as tiny granules insoluble in water. These granules are preserved in starches at temperatures below the initial gelatinization temperature. When put in cold water, the grains may absorb a small amount of the liquid. Up to 50° C. to 70° C. the swelling is reversible, the degree of reversibility being dependent upon the particular starch. With higher temperatures an irreversible swelling called gelatinization begins.

The term “initial gelatinization temperature” is understood as the lowest temperature at which gelatinization of the starch commences. Starch heated in water begins to gelatinize between 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given starch is the temperature at which birefringence is lost in 5% of the starch granules using the method described by Gorinstein. S. and Lii. C., Starch/Stärke, Vol. 44 (12) pp. 461-466 (1992).

The starch-containing material may also consist of or comprise a side stream from starch processing, e.g., C₆ carbohydrate containing process streams that may not be suited for production of syrups. In other embodiments, the starting material does not consist of or comprise a side stream from starch processing.

Reducing the Particle size of Starch-Containing Material

The starch-containing starting material may in a preferred embodiment be reduced in particle size prior to liquefaction. In a preferred embodiment the material is milled. Grinding is also understood as milling. Two kinds of milling are normally used: wet and dry milling. The term “dry milling” denotes milling of the starch-containing material using, e.g., a hammer or roller mill. In the case of using whole grain milling the whole kernel is milled and used in the remaining part of the process. Wet milling gives a good separation of germ and meal (starch granules and protein) and is often applied at locations where there is a parallel production of syrups. Other size reducing technologies such as emulsifying technology rotary pulsation may also be used.

### PROCESS OF THE INVENTION

The process of the present invention can generally be divided into the following main process stages: milling, in order to open up the structure of the starch-containing material and allowing for further processing; liquefaction, where the milled starch-containing material is hydrolyzed (broken down) to maltodextrins (dextrins); separate or simultaneous saccharification and fermentation, to produce low molecular fermentable sugars from maltodextrins that can be metabolized by the fermenting organism in questions, such as yeast, and converted into the desired fermentation product, such as ethanol; and optionally recovery, by, e.g., distillation to purify the desired fermentation product.

The individual process steps of fermentation product production, such as ethanol production may be performed batch wise or as a continuous flow process. For processes where all process steps are performed batch wise, or processes where all process steps are performed as a continuous flow, or processes where one or more process step(s) is(are) performed batch wise and one or more process step(s) is(are) performed as a continuous flow, are equally contemplated.

The cascade process is an example of a process where one or more process step(s) is(are) performed as a continuous flow and as such contemplated for the invention. For further information on the cascade process and other especially ethanol processes consult The Alcohol Textbook. Ethanol production by fermentation and distillation. Eds. T. P. Lyons, D. R. Kesall and J. E. Murtagh. Nottingham University Press 1995.

In the first aspect the present invention provides a process of producing a fermentation product, especially ethanol, from milled starch-containing material, preferably based on whole grain, comprising the steps of:

i) subjecting starch-containing material to an alpha-amylase,

ii) subjecting the material obtained in step i) to an alpha-glucosidase and optionally a glucose-generating and/or maltose-generating enzyme, and

iii) fermenting the material in the presence of a fermenting organism.

In a preferred embodiment the alpha-glucosidase is derived from a plant, preferably rice, especially rice (Oryzae sativa).

The present invention also relates to a process of producing a fermentation product from starch-containing material, which process comprises:

i) subjecting starch-containing material to an alpha-amylase,

ii) subjecting the material obtained in step i) to an alpha-glucosidase and E maltose-generating enzyme, and

iii) fermenting the material in the presence of a fermenting organism.

The starch-containing material as defined above in the “Raw Materials”-section is reduced in particle size before liquefaction step i). In a preferred embodiment the starch-containing material is milled. In a particular embodiment, the process of the invention further comprises, prior to the step i), the steps of:

x) reducing the particle size of starch-containing material;

y) forming a slurry comprising the starch-containing material and water.

The aqueous slurry may contain from 10-40 wt-%, preferably 25-35 wt-% starch-containing material. In one embodiment of the invention the slurry is heated to above the gelatinization temperature, such as between 60-95° C., preferably 80-85° C., and bacterial and/or acid fungal alpha-amylase may be added to initiate liquefaction (thinning). However, this is not mandatory.

The slurry of starch-containing material may in an embodiment be jet-cooked to further gelatinize the starch at 90-120° C., preferably around 105° C., for 1-15 minutes, preferably for 3-10 minute, especially around 5 minutes, before being subjected to an alpha-amylase in step i) of the invention. In a preferred embodiment the liquefaction in step i) is carried out by (a) treating the starch-containing material with, e.g., a bacterial alpha-amylase at a temperature around 70-90° C. for 15-120 minutes. Step (a) may be followed by a step (b) treating the material obtained in step (a) with an alpha-amylase at a temperature between 50-80° C. for 30-90 minutes. The alpha-amylase may be any alpha-amylase, including the ones mentioned in the “Alpha-Amylase”-section below. Preferred alpha-amylases are acid alpha-amylases. Liquefaction is performed at a pH in the range of about pH 4-7, preferably pH about 4.5-6.5. Whether the pH in the slurry is adjusted or not depends on the properties of the enzyme(s) used. Thus, in one embodiment the pH is adjusted, e.g., about 1 unit upwards, e.g., by adding NH₃. The adjusting of pH is advantageously done at the time when the alpha-amylase is added. In a preferred embodiment, the pH is not adjusted and the alpha-amylase has a corresponding suitable pH-activity profile, such as being active at a pH about 4. The liquefied whole grain is also known as mash.

In step ii) of the process of the invention the liquefied material, comprising maltodextrins, are hydrolyzed into low molecular fermentable sugars that can be metabolized by a fermenting organism, such as yeast. This step is referred to as “saccharification”. According to the present invention this step is carried out by subjecting the liquefied maltodextrin containing material to an alpha-glucosidase and a maltose-generating enzyme. The maltose-generating enzyme degrades the maltodextrins into maltose and the maltose is finally degraded by the alpha-glucosidase into glucose, which is consumed and converted into the fermentation product, e.g., ethanol, by the fermenting organism, e.g., yeast.

A full saccharification step may last up to 72 hours. However, the saccharification and fermentation (SSF) may in a preferred embodiment be combined, and in an embodiment of the invention a pre-saccharification step of 1-4 hours may be included. Pre-saccharification may be carried out at any suitable process conditions. In a preferred embodiment, the pre-saccharification is carried out at temperatures from 30-65° C., such as around 60° C., and at a pH, e.g., in the range from 4 to 5, especially around pH 4.5.

Thus, in one embodiment the method of the invention may further comprise a pre. saccharification step, as described herein, which is performed after the liquefaction in step i) and before step ii).

In a preferred embodiment a simultaneous saccharification and fermentation (SSF) process is employed where there is no holding stage for the saccharification, meaning that yeast and saccharification enzymes are added essentially together.

The invention also relates to a process of producing a fermentation product from starch-containing material, which process comprises:

a) subjecting starch-containing material to an alpha-glucosidase and optionally a glucose-generating and/or maltose-generating enzyme, and

b) fermenting in the presence of a fermenting organism.

In a preferred embodiment the fermentation product, such as especially ethanol, is recovered after fermentation, preferably by distillation. In a preferred embodiment step a may be preceded by pre-treatment at a temperature below the gelatinization temperature. According to this aspect of the invention the starch-containing is preferably raw granular starch. The starch may be of any plant origin as disclosed below in the “Raw Material” section. The alpha-glucosidase, glucose-generating enzyme, and maltose generating enzyme may be any of the enzymes disclosed in the “Enzyme Activities”-section below. Ir one embodiment the starch-containing material may further be subjected to an alpha amylase in step (a) and/or (b) and/or before step a). The alpha-amylase may be any of the alpha-amylases disclosed in the “Alpha-Amylase”-section below. Preferred are acid alpha amylases, especially of fungal origin.

Preferably the alpha-glucosidase, preferably derived from rice Oryzae sativa, is applied in a process, such as ethanol process, for saccharification of a gelatinized or a granular starch, said process comprising simultaneous saccharification and fermentation (SSF) and optionally recovery of the fermentation product. The SSF may be preceded by a gelatinization step, e.g., by jet cooking, or the SSF may be preceded by pre-treatment of raw granular starch at a temperature below the gelatinization temperature in order to achieve a swelling of the starch granules. In one embodiment step (a) is carried out below the initial gelatinization temperature as defined in the “Raw Materials”-section. Step (a) and (b) may be carried out sequentially or simultaneously. In a particular embodiment, the process of the invention further comprises, prior to the step a), the steps of:

x) reducing the particle size of starch-containing material;

y) forming a slurry comprising the starch-containing material and water.

The aqueous slurry may contain from 10-40 wt-%, preferably 25-35 wt-% starch-containing material. The slurry may include water and process waters, such as stillage (backset), scrubber water, evaporator condensate or distillate, side stripper water from distillation, or other fermentation product plant process water. Because the process is carried out below the gelatinization temperature and thus no significant viscosity increase takes place high levels of stillage may be used if desired. In an embodiment the aqueous slurry contains from about 1 to about 70 vol.-% stillage, preferably 15-60% vol.-% stillage, especially from about 30 to 50 vol.-% stillage.

The alpha-glucosidase may be applied alone or in combination with another amylolytic enzyme selected from the group comprising glucoamylase, amylases, including bacterial alpha-amylase, acid fungal alpha-amylase, beta-amylase, and pullulanase. In a preferred embodiment the alpha-glucosidase is applied in a process for hydrolysis of raw starch as disclosed in Danish patent application No. PA 2003 00812, WO 2004/106533 or WO 2004/081193, which are all hereby incorporated by reference. In another preferred embodiment the alpha-glucosidase is applied in a process for saccharification of a mash for beer production, said beer mash comprising starchy material selected from the group consisting of grain, rice, corn, wheat, barley, malt, unmalted barley, adjunct, non-grain adjunct and non-barley adjunct.

Fermentation

The term “fermenting organism” refers to any organism suitable for use in a desired fermentation process. Suitable fermenting organisms are according to the invention capable of fermenting, i.e., converting, preferably DP₁₋₃ sugars, such as especially glucose and maltose, directly or indirectly into the desired fermentation product, such as ethanol. The fermenting organism is typically added to the mash.

Examples of fermenting organisms include fungal organisms, such as yeast or filamentous fungi. Preferred yeast includes strains of the Saccharomyces spp., and in particular Saccharomyces cerevisiae. Commercially available yeast includes, e.g., RED STAR®/Lesaffre Ethanol Red (available from Red Star/Lesaffre, USA), FALI (available from Fleischmann's Yeast, USA) SUPERSTART (available from Alltech), GERT STRANC (available from Gert Strand AB, Sweden) and FERMIOL (available from DSM Specialties).

The fermentation is ongoing until the desired amount of fermentation product, such as ethanol, is produced. This typically means carrying out fermentation for 24-96 hours, such as 35-60 hours. The temperature and pH during fermentation is a temperature and pH suitable for the fermenting organism in question. For yeast, e.g., the temperature and pH is in the range about 26-34° C., preferably about 32° C., and the pH, e.g., is in the range about pH 3-6, e.g. about pH 4-5.

Preferred yeast for ethanol production includes, e.g., Pichia and Saccharomyces. Preferred yeast according to the invention is Saccharomyces species, in particular Saccharomyces cerevisiae or bakers yeast.

Recovery

The process of the invention may optionally comprise recovering the fermentation product, such as ethanol; hence the fermentation product, e.g., ethanol, may be separated from the fermented material and purified. Following fermentation, the mash may be distilled to extract, e.g., ethanol. Ethanol with a purity of up to, e.g., about 96 vol. % ethanol can be obtained by the process of the invention.

Thus, in one embodiment, the fermentation in step iii) and a distillation step may be carried out simultaneously and/or separately/sequentially; optionally followed by one or more process steps for further refinement of the fermentation product, e.g., ethanol.

Enzyme Activities

Alpha-Amylase

A process of the invention may be carried out in the presence of preferably, e.g., a bacterial and/or fungal alpha-amylase. Examples of suitable alpha-amylases include the below mentioned.

Bacterial Alpha-Amylases

Preferred bacterial alpha-amylases used, e.g., in step i) or step (a) of the invention, may be derived from a strain of B. licheniformis, B. amyloliquefaciens, B. stearothermophilus, or Bacillus subtilis. Also preferred are alpha-amylases having an amino acid sequence which has at least 50% homology, preferably at least 60%, 70%, 80%, 85% or at least 90%, e.g. at least 95%, 97%, 98%, or at least 99%, such as 100% homology to the sequences set forth in SEQ ID NO:2 or SEQ ID NO:3 herein.

Other bacterial alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 (or SEQ ID NO: 2 herein) or deletion of amino acids R179 and G180 using SEQ ID NO:3 in WO 99/19467 (or SEQ ID NO: 2 herein) for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted 1181*+G182*+N193F) compared to the wild-type BSG alpha-amylase amino acid sequence set forth in SEQ ID NO:3 disclosed in WO 99/19467 (or SEQ ID NO: 2 herein).

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with the following substitution: G48A+T491+G107A+H156Y+A181T+N190F+I201F+A209V+Q264S (using the numbering in SEQ ID NO: 4 of WO 99/19467) shown herein as SEQ ID NO:4. Also preferred are alpha-amylase variants derived from Bacillus amyloliquefaciens and having at least 50% homology, such as at least 60%, at least 70%, at least 80%, or even 90% homology to the sequence set forth in SEQ ID NO:4. Especially preferred are variants having one or more of the mutations H154Y, A181T, N190F, A209V and Q264S and/or deletion of two residues between positions 176 and 179, preferably deletion of E178 and G179 (using the SEQ ID NO: 5 numbering of WO 99/19467).

Other contemplated bacterial alpha-amylases are KSM-K36 alpha-amylase disclosed in EP 1,022,334 and deposited as FERM BP 6945 and KSM-K38 alpha-amylase disclosed in EP 1,022,334, and deposited as FERM BP-6946. Also variants therefore are contemplated, in particular the variants disclosed in WO 02/31124 (from Novozymes A/S.

Commercially available bacterial alpha-amylase products and products containing alpha-amylases include TERMAMYL™ SC and LIQUOZYME™ SC, BAN (Novozymes A/S, Denmark) and DEX-LO™, SPEZYME™ AA, and SPEZYME™ DELTA AA (from Genencor Int.)

Fungal Alpha-Amylases

Preferred fungal alpha-amylases are derived from a strain of Aspergillus, including Aspergillus oryzae, Aspergillus niger, or A. kawashii. Specifically contemplated are the Aspergillus oryzae TAKA alpha-amylase (EP 238 023); the Aspergillus niger alpha-amylase disclosed in EP 383,779 B2 (section [0037] (see also the cloning of the A. niger gene in Example 1); the Aspergillus niger alpha-amylase disclosed in Example 1 of EP 140,410. In a preferred embodiment the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. Such commercially available acid fungal amylase is SP288 (available from Novozymes A/S, Denmark).

The term “acid alpha-amylase” means an alpha-amylase (E.C. 3.2.1.1) which added in an effective amount has optimum activity at a pH in the range of 3.0 to 7.0, preferably from 3.5 to 6.0, or more preferably from 4.0-5.0.

A preferred acid fungal alpha-amylase is a Fungamyl-like alpha-amylase. In the present disclosure, the term “Fungamyl-like alpha-amylase” indicates an alpha-amylase which exhibits a high identity, i.e., more than 50%, 55%, 60%, 65%, 70%, 75%, 80%, 85% 90%, 95 or even more than 99% identical to the amino acid sequence shown in SEQ ID NO: 10 in WO 96/23874.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variants of said acid fungal amylase having at least 70% identity, such as at least 80% or even at least 90%, 95%, 96%, 97%, 98% or 99% identity thereto are contemplated. In an embodiment the acid fungal alpha-amylase is the one disclosed in SEQ ID NO: 1 herein, or a sequence being at least 70% identical, preferably at least 75%, at least 80%, at least 85% or at least 90%, e.g., at least 95%, at least 97%, at least 98%, or at least 99% identity to SEQ ID NO:1.

Commercial fungal alpha-amylases FUNGAMYL® (Novozymes A/S); and CLARASE™ (from Genencor Int., USA), the later both derived from Aspergillus.

Maltose Generating Enzymes

The maltose-generating enzymes used in a process of the invention may be a maltogenic amylase, a beta-amylases or a fungal alpha-amylase.

Maltogenic amylases (glucan 1,4-alpha-maltohydrolase) are able to hydrolyse amylose and amylopectin to maltose in the alpha-configuration. Furthermore, a maltogenic amylase is able to hydrolyse maltotriose as well as cyclodextrins. Specifically contemplated maltogenic amylases may be derived from Bacillus sp., preferably from Bacillus stearothermophilus, most preferably from Bacillus stearothermophilus C599 such as the one described in EP120.693. This particular maltogenic amylase has the amino acid sequence shown as amino acids 1-686 of SEQ ID NO:1 in U.S. Pat. No. 6,162,628. A preferred maltogenic amylase has an amino acid sequence having at least 70% identity to amino acids 1-686 of SEQ ID NO:1 in U.S. Pat. No. 6,162,628, preferably at least 80%, at least 85%, at least 90%, at least 92%, at least 95%, at least 96%, at least 97%, at least 98%, or particularly at least 99%. Most preferred variants of the maltogenic amylase comprise the variants disclosed in WO99/43794.

Maltogenic amylases may be added in amounts of 0.01-40.0 MANU/g DS, preferably from 0.02-10 MANU/g DS, preferably 0.05-5.0 MANU/g DS.

Another maltose generating enzyme to be used in the processes of the invention may be a beta-amylase (E.C 3.2.1.2). Beta-amylase is the name traditionally given to exo-acting maltogenic amylases, which catalyze the hydrolysis of 1,4-alpha-glucosidic linkages in amylose, amylopectin and related glucose polymers.

Beta-amylases have been isolated from various plants and micro-organisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7.0. Preferably the beta-amylase is derived from a filamentous fungus, such as a beta-amylase derived from Rhizomucor pusilis. Contemplated beta-amylase include the beta-amylase from barley SPEZYME® BBA 1500, SPEZYME® DBA and OPTIMALT™ ME, OPTIMALT™ BBA from Genencor Int. as well as NOVOZYM™ WBA from Novozymes A/S.

Another maltose generating enzyme which may be used in a process of the invention is a fungal alpha-amylase (EC 3.2.1.1), such as a fungamyl-like alpha-amylase. In the present disclosure, the term “fungamyl-like alpha-amylase” indicates an alpha-amylase which exhibits a high homology, i.e. more than 50%, 55%, 60%, 65%, 70%, 75%, 80%, 85%, 90%, 95%, 96%, 97%, 98% or even 99% homology (identity) to the amino acid sequence shown in SEQ ID No. 10 in WO 96/23874.

When used as a maltose-generating enzyme fungal alpha-amylases may be added in an effective amount, preferably of from 0.001-1.0 AFAU/g DS, preferably from 0.002-0.5 AFAU/g DS, preferably 0.02-0.1 AFAU/g DS or preferably 0.01-10 mg protein/g DS of maltogenic amylase, beta-amylase, Fungamyl-like alpha-amylase, or mixtures thereof.

Alpha-Glucosidases

An alpha-glucosidase or maltase (EC 3.2.1.48) used in a process of the invention may be derived from a micro-organism or a plant. Preferred is alpha-glucosidases of fungal origin, such as an alpha-glucosidase derived from yeast or from a filamentous fungi, and of bacterial origin. A preferred fungal alpha-glucosidase is one derived from a strain of Candida sp. such as a strain of C. edax, preferably the strain CBS 6461. Also preferred are the alpha-glucosidases derivable from a strain of Pichia sp., such as a strain of P. amylophilia, P. missisippiensis, P. wicherhamii and P. rhodanensis. Also contemplated are alpha-glucosidases derived from Aspergillus sp, such as A. nidulans (Kato et al. 2002, Appl Environ Microbiol. 68: 1250-1256), from Rhizobium sp. (Berthelot et al. 1999, Appl Environ Microbiol. 65: 2907-2911).

Preferred bacterial alpha-glucosidases include alpha-glucosidases derived from the genus Bacillus, such as from a strain of Bacillus stearothemophilus. Preferred are alpha-glucodsidases having an amino acid sequence which has at least 50% homology(identity), preferably at least 60%, at least 70%, at least 80%, at least 85% or at least 90%, e.g., at least 95%, at least 97%, at least 98%, or at least 99%, such as 100% homology (identity) to the mature sequence set forth in SEQ ID NO:6 herein. A commercially available alpha-glucosidase contemplated is the Bacillus stearothemophilus alpha-glucosidase commercially available from SIGMA (Sigma cat. No. G3651). Alpha-glucosidases of plant origin may be derived from a cereal, such as from wheat, rye, barley corn or rice. Other alpha-glucosidases contemplated include Aspergillus fumigatus alpha-glucosidases, especially the ones disclosed in U.S. patent application No. 60/585,336 or Fusarium venenatum alpha-glucosidases, especially the ones disclosed in U.S. patent application No. 60/586,103 (both application hereby incorporated by references).

A preferred plant alpha-glucosidase is derived from rice, e.g. Oryzae sativa. Preferably the alpha-glucosidase has the N-terminal amino acid sequence; GYNVASVAGS (SEQ ID NO: 7), more preferably the alpha-glucosidase has the N-terminal amino acid sequence; GYNVASVAGS KNRRRARREL AAGGGGA (SEQ ID NO:8), or the alpha-glucosidase has an N-terminal amino acid sequence comprising an amino acid sequence corresponding to any of the two aforementioned amino acid sequences wherein preferably no more than one, more preferably no more than two, even more preferably no more than three, and most preferably no more than four amino acid residues have been substituted, inserted and/or deleted. A preferred rice alpha-glucosidase is available from Sigma-Aldrich as Cat. No. G9259. Also preferred is the rice alpha-glucosidase disclosed in Iwata et al. in Journal of Bioscience and Bioengineering, Vol. 95, No. 1, 106-108.2003. Preferably the alpha-glucosidase has a MW of approximately 90 kDA to 100 kDa, more preferably of approximately 92 kDa to 99 kDa, such as from approximately 95 kDa to 98 kDa. A particularly preferred alpha-glucosidase has a MW of approximately 97 kDa.

Alpha-glucosidase may be added an effective amount of 0.1-10000 maltase units/kg DS, 1-1000 maltase units/kg DS, or more preferably 10-100 maltase units/kg DS, such as or more preferably 1-10 maltase units/kg DS or preferably from 0.01 to 10 mg protein/g DS or 0.001 to 100 mg protein/g DS, preferably from 0.01 to 10 mg protein/g DS.

Glucose Generating Enzymes

Any glucose-generating enzymes may be used according to the invention. The preferred glucose-generating enzyme is a glucoamylase. The glucoamylase may be of any origin, e.g., derived from a micro-organism or a plant. Preferred is glucoamylase of fungal or bacterial origin selected from the group consisting of Aspergillus niger glucoamylase, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other contemplated Aspergillus glucoamylase variants include variants to enhance the thermal stability: G137A and G139A (Chen et al. (1996), Prot. Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Furthermore, Clark Ford presented a paper on Oct. 17, 1997, ENZYME ENGINEERING 14, Beijing/China October 12-17, 97, Abstract number: Abstract book p. 0-61. The abstract suggests mutations in positions G137A, N20C/A27C, and S30P in an Aspergillus awamori glucoamylase to improve the thermal stability.

Other glucoamylases include Talaromyces glucoamylases, in particular derived from Talaromyces emersonii (WO 99/28448), Talaromyces leycettanus (U.S. Pat. No. Re. 32,153), Talaromyces duponti, Talaromyces thermopiles (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Also glucoamylases derived from Athelia rolfsii (previously denoted Corticium rolfsii) are specifically contemplated, including the one having the amino acid sequence available as SPTREMBL:Q12596. See also U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330).

Commercial available products comprising a glucoamylase include SPIRIZYME™ FUEL, SPIRIZYME PLUS, SAN™ SUPER™ and AMG™ E (from Novozymes A/S).

A glucoamylase may be added in an effective amount, preferably 0.02-20 AGU/g DS, preferably from 0.005 to 5 AGU/g DS, or 0.1-10 AGU/g DS, preferably 0.05 to 0.5 AGU/g DS such as around 0.1, 0.3, 0.5, 1 or 2 AGU/g DS, such as between 0.1-0.5 AGU/g DS.

Pullulanase

Pullulanases (E.C. 3.2.1.41, pullulan 6-glucano-hydrolase), are de-branching enzymes characterized by their ability to hydrolyze the alpha-1,6-glycosidic bonds in, for example, amylopectin and pullulan.

Specifically contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 and SEQ ID NO: 11 in U.S. Pat. No. 5,736,375 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

The pullulanase may according to the invention be added in an effective amount which include the preferred range of from between 1-100 micro g per g DS, especially from 10-60 micro g per g DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

Use of the Products Produced by the Process of the Invention

Ethanol obtained by the process of the invention may be used as, e.g., fuel ethanol; drinking ethanol, i.e., potable neutral spirits, or industrial ethanol, including fuel additive.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

Material & Methods

Enzymes:

Bacterial Alpha-Amylase A (BAAA): Bacillus stearothermophilus alpha-amylase variant with the mutations: 1181*+G182*+N193F disclosed in U.S. Pat. No. 6,187,576 and available on request from Novozymes A/S, Denmark.

Fungal acid alpha-amylase B (FMB): Aspergillus niger alpha-amylase disclosed in SEQ ID NO: 1 and available from Novozymes A/S.

Alpha-glucosidase BS (AGBS): Bacillus stearothemophilus alpha-glucosidase available from SIGMA (Sigma cat. No. G3651)

Maltose generating enzyme: Maltogenic amylase derived from Bacillus stearothermophilus C599 described in EP120.693 and available from Novozymes A/S.

Alpha-glucosidase OS: Oryzae sativa alpha-glucosidase available from SIGMA (Sigma cat. No. G9259).

Glucoamylase TN: Glucoamylase derived from Talaromyces emersonii and disclosed as SEQ ID NO: 7 in WO 99/28448 with side activity of Aspergillus niger glucoamylase and As-pergillus niger acid alpha-amylase.

Pullulanase PD: Pullulanase derived from Bacillus deramificans having the amino acid sequence shown as SEQ ID NO:11 in U.S. Pat. No. 5,736,375 and disclosed as SEQ ID NO: 9 herein.

Beta-amylase WG: A plant beta-amylase extracted from wheat grain (Novozym® WBA available from Novozymes A/S).

Determination of Homology (Identity)

The term polypeptide “homology” means the degree of identity between two amino acid sequences. The homology may suitably be determined by computer programs known in the art, such as, GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C.D., (1970), Journal of Molecular Biology, 48, 443-453. The following settings for polypeptide sequence comparison are used: GAP creation penalty of 3.0 and GAP extension penalty of 0.1.

Determination of Alpha-Amylase Activity (KNU)

The KNU is used to measure bacterial alpha-amylases with high pH optima.

PHADEBAS™ Assay

Alpha-amylase activity is determined by a method employing PHADEBAS tablets as substrate. PHADEBAS tablets (Phadebas® Amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temp., pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

Alternative Method

Alpha-amylase activity is determined by a method employing the PNP-G₇ substrate. PNP-G₇ which is an abbreviation for p-nitrophenyl-alpha,D-maltoheptaoside is a blocked oligosaccharide which can be cleaved by an endo-amylase. Following the cleavage, the alpha-Glucosidase included in the kit digest the substrate to liberate a free PNP molecule which has a yellow colour and thus can be measured by visible spectophometry at lambda=405 nm. (400-420 nm.). Kits containing PNP-G₇ substrate and alpha-glucosidase is manufactured by Boehringer-Mannheim (cat. no.1054635).

To prepare the substrate one bottle of substrate (BM 1442309) is added to 5 ml buffer (BM1442309). To prepare the alpha-glucosidase one bottle of alpha-glucosidase (BM 1462309) is added to 45 ml buffer (BM1442309). The working solution is made by mixing 5 ml alpha-glucosidase solution with 0.5 ml substrate.

The assay is performed by transforming 20 microL enzyme solution to a 96 well microtitre plate and incubating at 25° C. 200 micro I working solution, 25° C. is added. The solution is mixed and pre-incubated 1 minute and absorption is measured every 15 sec. over 3 minutes at OD 405 nm.

The slope of the time dependent absorption-curve is directly proportional to the specific activity (activity per mg enzyme) of the alpha-amylase in question under the given set of conditions.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

Determination of Acid Alpha-Amylase Activity (AFAU)

Acid alpha-amylase activity is measured in AFAU (Acid Eungal Alpha-amylase Units), which are determined relative to an enzyme standard.

The standard used is AMG 300 L (from Novozymes A/S, glucoamylase wildtype Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. I FAU/mL to below 0.05 FAU/mL.

The acid alpha-amylase activity in this AMG standard is determined in accordance with the following description. In this method, I AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

| | | |
|---|---|---|
| | Alpha-amylase | |
| Starch + Iodine | → | Dextrins + Oligosaccharides |
| | 40° C., pH 2.5 | |
| Blue/violet | t = 23 sec. | Decoloration |

Standard Conditions/Reaction Conditions: (per Minute)

| Substrate: | Starch, approx. 0.17 | g/L |
|---|---|---|
| Buffer: | Citate, approx. 0.03 | M |
| Iodine (I₂): | 0.03 | g/L |
| CaCl₂: | 1.85 | mM |
| pH: | 2.50 + 0.05 | |
| Incubation temperature: | 40° | C. |
| Reaction time: | 23 | seconds |
| Wavelength: | lambda = 590 | nm |
| Enzyme concentration: | 0.025 | AFAU/mL |
| Enzyme working range: | 0.01-0.04 | AFAU/mL |

If further details are preferred these can be found in EB-SM-0259.02/01 available on request from Novozymes A/S, and incorporated by reference.

Alpha-Glucosidase Activity (Maltase Units)

The alpha-glucosidase activity can be expressed in maltase units (g glucose formed/L maltase preparation/hour). A maltase preparation is incubated at 60° C. in a 20% w/v maltose solution, in 50 mM citrate at pH=4.5 for 60 minutes (1 hour). The amount of glucose liberated is measured using the GOD-PERID assay, Boehringer Mannheim.

Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan. 1 ml diluted sample or standard is incubated at 40° C. for 2 minutes. 0.5 ml 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### EXAMPLES

### Example 1

### Liquefaction with Bacterial Acid Alpha-Amylase

100 mL of milled corn slurry is liquefied with 50 NU/g dry solids (DS) of Bacterial Alpha-Amylase A from Bacillus stearothermophilus. The corn mash has about 30% dry substance (pH 5.4). The mash is heated to 85° C. for 0.5 hour. The temperature is then lowered to 70° C. and mash is then treated with acid fungal alpha amylase B from Aspergillus niger having the amino acid sequence disclosed in SEQ ID NO:1. The enzyme loading is 0.05 AFAU/g dry solids. After 1.0 hours samples are taken for HPLC analysis. Temperature is brought down to 32° C. for carrying out simultaneous saccharification and fermentation (SSF).

SSF with Glucoamylase, Alpha-Glucosidase and Maltose-Generating Enzyme

Once the liquefaction is over, pH is adjusted to 5.0. The mash is then treated with Aspergillus niger glucoamylase (0.1 AGU/g DS) including a side activity of Aspergillus niger acid alpha-amylase (the ratio between AGU and AFAU is approx. 9:1), Alpha-Glucosidase BS (equivalent glucoamylase protein dose of 0.1 AGU/g DS) and a maltogenic amylase (equivalent glucoamylase protein dose of 0.1 AGUI g DS). The mash is then inoculated with yeast (Saccharomyces cerevisiae) (4% w/w) and incubated at 32° C. for the complete course of fermentation. Samples are taken at regular interval to perform HPCL for ethanol and sugar profile.

### Example 2

A 33% dry solids (DS) whole corn mash was liquefied in a three-step hot slurry process using 50 NU/g DS of Bacterial Alpha-Amylase A from Bacillus stearothermophilus. First the slurry was heated to about 82° C. and one third (⅓) of the alpha-amylase was added to initiate liquefaction. Then the slurry was jet-cooked at a temperature of about 112° C. to complete gelatinization of the slurry. Then the slurry was cooled to about 77° C. and the remaining two thirds (⅔) of the alpha-amylase were added to finalize hydrolysis. 250 mL liquefied whole corn mash was filled into a 500 mL blue cap bottles with magnetic stirrers. The pH of the mash was adjusted to about 5.5. The bottles were incubated in a water bath at about 32° C. for 40 minutes before dry yeast (Saccharomyces cerevisiae) was added at a dosage of 0.2 g/bottle equivalent to 15 mill cells viable count per mL. The bottles were closed using a yeast-lock filled with concentrated H₂SO₄. The fermentation at 32° C. was continued for about 91 hours and by weighing the bottle at regular intervals the CO₂ loss, which is proportional to the ethanol production, was monitored.

TABLE 1
Results of SSF performed without alpha-glucosidase (Ag) (A and
B) and with alpha-glycosidase (C or D) shown as weight loss in
grams. Treatment A received Glucoamylase TN (0.5 AGU/g DS), 33%
DS, all other treatments received Glucoamylase TN (0.05 AGU/g
DS) + Fungal acid alpha-amylase B (0.0125 mg protein/g DS) +
Pullulanase PD (0.0125 mg protein/g DS). The alpha-glucosidases
was dosed as 0.0125 mg protein/g DS.

| | | | C | D |
|---|---|---|---|---|
| | | | Rice Ag 0.0125 mg | Rice Ag 0.0031 mg |
| Hours | A | B | protein/g DS | protein/g DS |
| 49.25 | 10.59 | 9.45 | 10.31 | 9.75 |
| 65.75 | 11.02 | 10.49 | 11.02 | 10.96 |
| 73.50 | 11.05 | 10.79 | 11.24 | 11.27 |
| 91.00 | 11.06 | 11.14 | 11.55 | 11.60 |

### Example 3

A 30% D.S. slurry of milled wheat grain is made in room temperature (RT) tap water. For each treatment 2×250 g are portioned in 500 mL blue cap fermentation flasks. The pH is adjusted to 6.0 and enzymes are added: Bacterial Alpha-Amylase A from Bacillus stearothermophilus alpha-amylase (0.15 KNU/g DS), Beta-amylase WG (0.0125 mg EP/g DS), and alpha-glucosidase from Oryzae sativa (0.0125 mg EP/g DS). A pre-treatment is carried out for 60 minutes at about 55° C. in a shaking water bath. The flasks are cooled to about 32° C., 0.25 g dry bakers yeast (corresponding to 10 million viable cells/g mash) is added to each flask, the flasks are equipped with air locks, and weighed. The flasks are incubated in a shaking water bath preset at about 32° C. and a simultaneous saccharification and fermentation (SSF) process step is carried out for 96 hours. The flasks are weighed at regular intervals and CO₂ weight loss (g) is measured for monitoring of the fermentation progress. The relationship used between amount of CO₂ loss and the weight of ethanol is:

$$Litre Et OH / 100 kg mash dry matter = \frac{Weight loss ( g ) ⨯ 1.045}{0.79 ( g \text{/} mL ) ⨯ 250 ⨯ 20 \% dry matter} ⨯ 100$$

### Example 4

The process described in Example 3 is repeated; except that the slurry is a 30% DS dry milled corn slurry.

### Example 5

### Effect of Adding Alpha-Glucosidase During SSF

This Example investigates the impact of alpha-glucosidase in combination with pullulanase, acid alpha-amylase and a low dosage of glucoamylase on the sugar, glycerol and ethanol profile over the complete span of SSF.

Two identical vessels (each 5 liters of total volume) were used to carry out the complete process including liquefaction and SSF. A working volume of approximately 2.5 kg was used. For liquefaction, a single reactor was used to have a common liquefied material. Ground corn was used to make a liquid slurry with 30 wt. % dry solids (DS) using tap water making up the final weight about 5.5 Kg. The pH was adjusted to 5.8 using diluted NaOH. Once the pH was adjusted, Bacterial Alpha-Amylase A (BAAA) (0.04% w/w of corn) was added to the vessel. After mixing the enzyme with corn slurry, temperature was raised to 85° C. by circulating hot water through jacket. After the temperature reached 85° C., it was held for 1.5 hours before cooling it down to 32° C. For rest of the course of the experiment the temperature was maintained at 32° C.

Once the liquefaction was complete, mash was divided in two fermentors equally. In the first reactor (fermentor 1), only glucoamylase (Glucoamylase TN) was added (0.5 AGU/g DS) as a Reference run. In the second reactor (fermentor 2, Test run), the glucoamylase (Glucoamylase TN) dose was reduced to 10% (compared to Reference run) making it to 0.05 AGU/g DS) along with 5 wt. % enzyme protein of original Glucoamylase TN dose for 3 enzymes (i.e., Fungal Acid Alpha-Amylase B (FAAAB), Pullulanase PD and Alpha-Glucosidase OS enzyme protein each equivalent to 0.025 AGU/g DS of Glucoamylase TN). Urea (1000 ppm) and penicillin (3 mg/L) were added to each fermentor based on the total mash weight. Finally, the reactors were inoculated with 0.04 mL/g mash of yeast propagate (RED STAR™) that had been grown for 20 hours. Agitation was maintained at 550 rpm in each vessel. Samples were taken with regular intervals and analyzed for sugars and ethanol profiles and for viable yeast count by plating on 3M Petrifilm. To minimize evaporation of ethanol and water during the fermentation, the off-gas was passed through a condenser where water at 2° C. was circulated.

Results

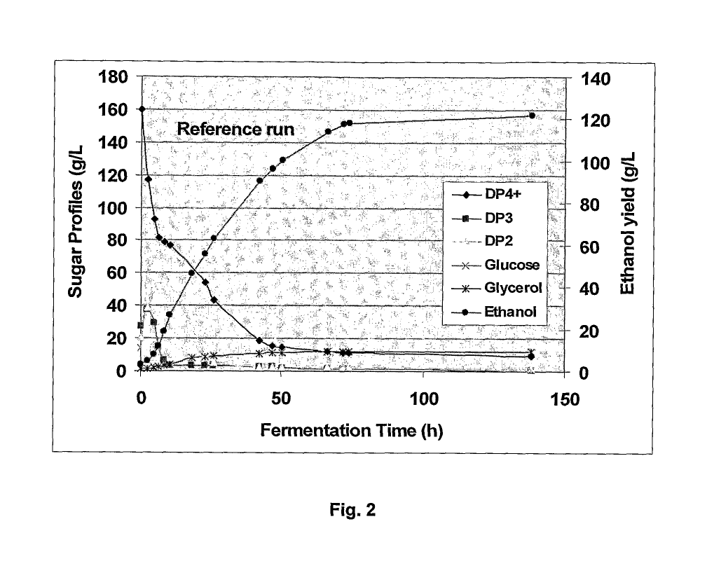

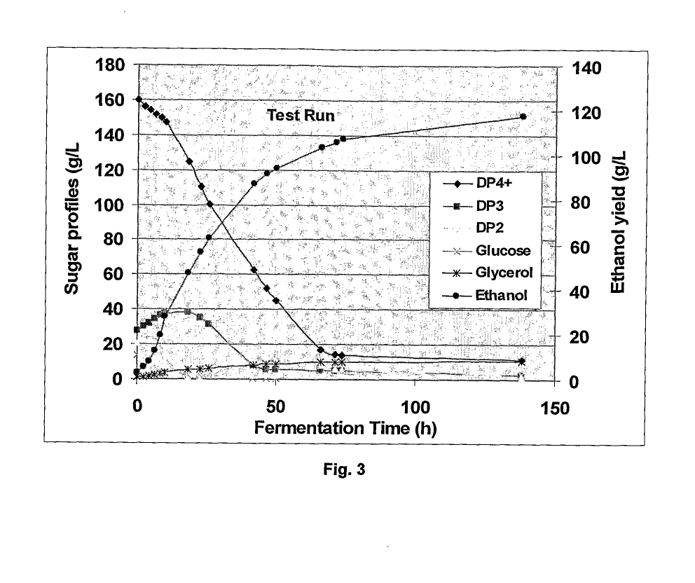

FIGS. 2 and 3 show the sugar, glycerol and ethanol profiles for the Reference and Test runs, respectively. As can be seen from the plots, the rate of DP4+ hydrolysis is relatively faster in the Reference run specifically in the initial 15 hours, which could be attributed to a significantly higher Glucoamylase TN activity.

Similar observation was seen with maltotriose (DP3). However, in case of maltose, for initial 25 hours, relative concentrations were found to be much lower in the Test run. This can be explained by the presence of alpha-glucosidase in the enzyme mixture.

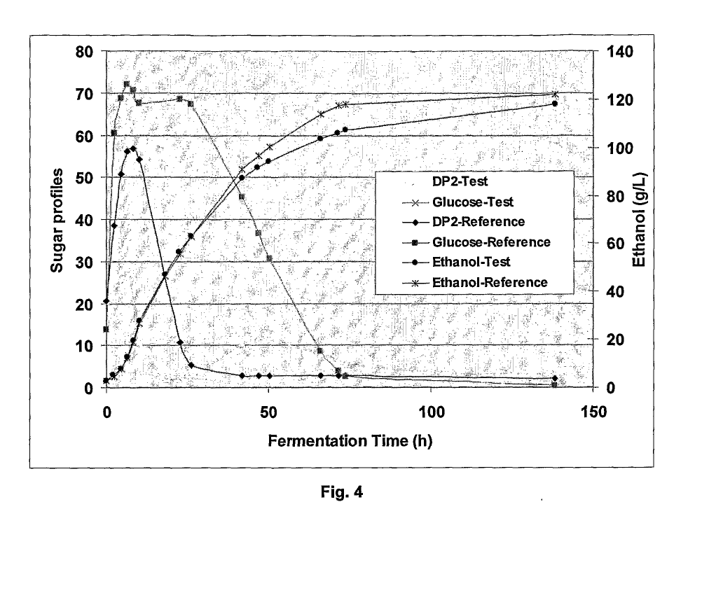

A slow DP4+ hydrolysis (a low dose of Glucoamylase TN) coupled with the exponential yeast growth, resulted in consumption of generated glucose relatively rapid in the Test run. However, a higher dose of Glucoamylase TN resulted in higher glucose release in case of Reference run (shown in FIG. 4).

Another important finding was the increase in maltose concentration after 40 hours in the Test run. One reason could be the de-activation of alpha-glucosidase due to the presence of ethanol which resulted in maltose accumulation, showing the importance of alpha-glucosidase in the sugar utilization pattern.

As shown in FIG. 4, the overall ethanol yield was found to be higher in Reference run than in the Test run. However, only 25 wt. % of the total enzyme protein was added in the Test run.

When looking at the glycerol concentrations, they ended at 10.7 g/L and 12.0 g/L for the Test and Reference run, respectively. This could be a representation of the stress on yeast, indicating that a synergetic sugar profile could result in better growth environment for yeast and better utilization of sugars.

In conclusion, a better utilization of glucose as well as maltose was obtained as no accumulation of any sugar was seen. This shows a synergy between the Test run enzymes. In addition, a lower glycerol generation was observed with the adding of an enzyme combination with alpha-glucosidase.

1-57. (canceled)

58. A process of producing a fermentation product from starch-containing material, which process comprises:
i) subjecting starch-containing material to an alpha-amylase,
ii) subjecting the material obtained in step i) with an alpha-glucosidase, and
iii) fermenting the material in the presence of a fermenting organism.

59. The process of claim 58, further comprising adding a glucose-generating and/or maltose-generating enzyme in step ii).

60. The process of claim 58, wherein the fermentation product is ethanol.

61. The process of claim 58, wherein the fermentation product is recovered after fermentation, preferably by distillation.

62. The process of claim 58, wherein the saccharification in step ii) and the fermentation in step iii) are carried out simultaneously (SSF).

63. The process of claim 58, wherein step i) is carried out by (a) treating the starch-containing material with an alpha-amylase at a temperature around 70-90° C. for 15-120 minutes.

64. The process of claim 63, wherein the wherein step i) (a) is followed by (b) treating the material obtained in step (a) with an alpha-amylase at a temperature between 50-80° C. for 30-90 minutes.

65. The process of claim 64, wherein the alpha-amylase in step (b) is an acid alpha-amylase derived from Aspergillus niger or Aspergillus oryzae.

66. The process of claim 64, wherein the starch-containing material is jet-cooked at 90-120° C. for 1-15 minutes before step i).

67. The process of claim 58, wherein the alpha-amylase in step i) is of bacterial origin.

68. The process of claim 58, wherein the alpha-amylase in step i) is a Bacillus alpha-amylase.

69. The process of claim 67, wherein the alpha-amylase is a Bacillus stearothermophilus alpha-amylase variant with mutations in 1181*+G182*.

70. The process of claim 59, wherein the maltose-generating enzyme is a beta-amylase and/or a maltogenic amylase.

71. The process of claim 59, wherein the glucose-generating enzyme is a glucoamylase.

72. The process of claim 58, wherein the alpha-glucosidase is derived from a plant.

73. The process of claim 58, wherein the alpha-glucosidase is derived from a strain of Bacillus stearothermophilus.

74. The process of claim 58, wherein the alpha-glucosidase is derived from rice (Oryzae sativa).

75. The process of claim 58, wherein the starch-containing material is selected from the group consisting of corn, cob, wheat, barley, rye, milo and potatoes; or any combination of these.

76. The process of claim 58, further comprising prior to step i) the steps of:
x) reducing the particle size of starch-containing material; and
y) forming a slurry comprising the material and water.

77. The process of claim 58, wherein the fermenting organism is yeast.

78. The process of claim 58, wherein the fermenting organism derived from a strain of Saccharomyces cerevisiae.
